(19) United States
(12) Patent Application Publication
(10) Pub. No.: US 2008/0208259 A1
(43) Pub. Date: Aug. 28, 2008

(54) LOCKING FIXATION SYSTEM AND LAG TOOL

(75) Inventors: Stephen GILBERT, West Chester, PA (US); Thomas LORING, Englewood, NJ (US); John MINIER, Highland Mills, NY (US)

Correspondence Address: MORGAN, LEWIS & BOCKIUS LLP, 1701 MARKET STREET, PHILADELPHIA, PA, 19103-2921, US

(73) Assignee: SMALL BONE INNOVATIONS, INC.

(21) Appl. No.: 11/960,648

(22) Filed: Dec. 19, 2007

Related U.S. Application Data
(60) Provisional application No. 60/875,689, filed on Dec. 19, 2006.
(60) Provisional application No. 60/975,936, filed on Sep. 28, 2007.

(51) Int. Cl. A61B 17/58; A61B 17/56

(52) U.S. Cl. 606/280; 606/300; 606/301

(57) ABSTRACT

The present application relates to a locking bone fixation device having tabs to engage the threads of an orthopedic fixation element. A tool and method for inducing compression across a fracture plane is also disclosed.

### CROSS-REFERENCE TO RELATED APPLICATIONS

This application claims the benefit of U.S. Provisional Application Nos. 60/875,689 filed on Dec. 19, 2006, and 60/975,936 filed on Sep. 28, 2007, the contents of which are hereby incorporated by reference in their entirety.

### BACKGROUND

Bone plates are commonly used to assist in the healing of broken and fractured bones, especially in situations where casts cannot be applied to the injured area (e.g., fractures occurring to facial areas such as the nose, jaw or eye sockets). A fractured or broken bone may first need to be reset into its proper position before the fracture site is held in place by a bone plate secured to the bone with a screw or other type of fastener.

Some bone plates have a locking mechanism to lock the fastener to the bone plate: e.g., those described in U.S. Pat. No. 5,601,553 to Trebling et al., U.S. Pat. No. 6,206,881 to Frigg, et al., and U.S. Pat. No. 6,974,461 to Wolter, each of which is hereby incorporated by reference in its entirety. The '553 and '881 patents describe a locking mechanism where threads on a screw head and threads on the plate match and are interlocked as the screw head is engaged. The '461 patent describes a method whereby a threaded screw head engages a non-threaded bone plate, such that the threads of the screw head tap a hole in the unthreaded plate.

However, both of these locking mechanisms have drawbacks. A disadvantage of the mechanism described in the '553 and '881 patents is the inability to induce compression (lag) across a fracture plane. In order to confer the benefit of an accelerated healing process and reduced recovery time, the bone plate not only needs to secure the break or fracture site in place, but also needs to maintain the break or fracture under some slight pressure for a sufficient length of time. In the mechanism taught in the '461 patent, the thread on the head of the screw is limited in its ability to engage the plate and is also associated with an increased risk of gauling (cold welding) of the screw to the bone plate. The potential for gauling of the screw to the plate may be minimized by requiring the material of the plate and that of the screw used to be of sufficiently different hardness. However, such a requirement imposes needless plate design constraints and leads to increased manufacturing costs.

Thus, there exists a need for a bone plate with an improved locking mechanism that desirably enables locking of the fastener therein and reliably provides sustained compression across a break or a fracture plane.

### SUMMARY OF THE INVENTION

The present invention relates generally to a fixation device for fixing two bones together, where the fixation system has locking fixation. The present invention also relates to a fixation system whereby at least one fixation element (e.g., a bone screw, a nail, or other fastener) engages a fixation device. The present invention includes a fixation device comprising a first outer surface, a second outer surface, and an aperture defined at least in part by an aperture wall. In one embodiment, the aperture extends from the first outer surface to the second outer surface. In one embodiment, the tabs protrude from the aperture wall into the aperture, and each of the tabs is configured to form a locking securement with a fixation element disposed within the aperture.

In one embodiment, the fixation element engages the fixation device at an angle. In one embodiment, the fixation element is configured to reduce interference upon the fixation device. In one embodiment, the fixation element is configured to engage the tabs of a fixation device and lock into place.

In one embodiment, the fixation device includes an angular surface. In another embodiment, the fixation device includes an angular surface and a substantially flat surface. In one embodiment, the fixation device includes at least one angular tab cut to facilitate insertion of a fixation element. In one embodiment, the fixation device is configured to lock the fixation element into place.

The present invention also relates to a fixation element having threading that includes a radiused profile. In one embodiment, the threading for the fixation elements includes a superior angular thread profile. In one embodiment, the treading for the fixation elements includes an inferior radiused thread profile. In another embodiment, the threading for the fixation elements includes both a superior angular and an inferior radiused thread profile.

The present invention also relates to an instrument useful in inducing compression, or lag, across a fracture plane. In one embodiment, the instrument is configured to advance a fixation element into a fracture plane without the head of the fixation element coming into contact with the fixation device until a desired compression is achieved across the fracture site being repaired.

The present invention also relates to a method for inducing compression including partially advancing a fully threaded or partially threaded locking fixation element into an aperture of a fixation device, placing the lag tool between the fixation element head and the fixation device, turning the fixation element until the desired amount of compression is achieved across a fracture site being repaired, removing the lag tool, and then locking the fixation element into the fixation device.

### BRIEF DESCRIPTION OF THE DRAWINGS

The foregoing summary, as well as the following detailed description of various embodiments of the invention, will be better understood when read in conjunction with the appended drawings. Drawings are provided for the purpose of illustrating certain embodiments of the invention. It should be understood, however, that the invention is not limited to the precise arrangements and instrumentalities shown, including the recited dimensions. In the drawings:

FIG. 1 depicts an example of a fixation element in accordance with one embodiment of the present invention.

FIGS. 2a and 2b illustrate a tab configuration in the aperture of a fixation device in accordance with an embodiment of the present invention.

FIGS. 3a and 3b are respectively an isometric and a cross-sectional view of a locking fixation system in accordance with one embodiment of the present invention.

FIGS. 4a and 4b are cross-sectional views of a fixation element engaging the aperture and tab of a fixation device in accordance with an embodiment of the present invention.

FIG. 5 is a diagram illustrating an angular profile for a fixation element thread.

FIG. 6 is a diagram illustrating a radiused profile according to the present invention.

FIG. 7 is an isometric illustration of a lag tool and locking fixation system according to the present invention.

FIG. 8 is a top view illustration of a lag tool and locking fixation system according to the present invention.

FIGS. 9a, 9b, and 9c are isometric illustrations of a lag tool according to the present invention.

FIG. 10 is a cross-sectional view of a fixation device including a tab with an angular superior surface and a substantially flat inferior surface in accordance with an embodiment of the present invention.

FIG. 11 is a cross-sectional view of a fixation device including a tab with a substantially flat superior surface and an angular inferior surface in accordance with an embodiment of the present invention.

FIG. 12 is a cross-sectional view of a fixation device including a tab with angular superior and inferior surfaces in accordance with an embodiment of the present invention.

FIG. 13 is a cross-sectional view of a fixation device with an angular tab slot in accordance with an embodiment of the present invention.

FIG. 14 is an illustration of the interaction of the tab with the fixation element head threads in accordance with an embodiment of the present invention.

### DETAILED DESCRIPTION

The present invention is described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Moreover, the present invention may be embodied as methods or devices. Accordingly, the present invention may take the form of an entirely mechanical device embodiment, an entirely method embodiment or an embodiment combining method and mechanical device aspects. The following detailed description is, therefore, not to be taken in a limiting sense.

Locking Fixation System

The present invention relates to a locking fixation system for repairing bone fractures. In certain embodiments, the locking fixation system lockingly engages a fixation element (i.e., a “fastener”) to a fixation device (e.g., a bone plate). Moreover, in certain embodiments, the fixation element head is configured to engage a fixation device at several different angles. Angular fixation improves stability of the fixation device over time and in the face of pressure and stresses on the particular bone or bones being fixed.

In an embodiment of the invention, the locking fixation system includes a fixation device and at least one fixation element, both of which are more fully described below. Any size fracture on any size bone may be fixed using the fixation system of the present invention. In an embodiment of the invention, one advantage of the fixation system of the present invention is the capability for angular stability. In an embodiment of the invention, angular stability minimizes the risk of a mal-union or a non-union, particularly with respect to articular or sub-articular fractures.

A. Fixation Device

A fixation device in accordance with the present invention and drawings is described herein. As used herein, the term “fixation device” means a device that fixes two or more parts together to create a union of such parts. In an embodiment of the present invention, the fixation device is a bone plate. In an embodiment of the invention, the bone plate is a regular shape. In an embodiment of the invention, the bone plate is a shape selected from the group consisting of a Y-shape, a T-shape, an L-shape, a straight plate, or a curved plate. In another embodiment, the bone plate is an irregularly shaped plate. In another embodiment, the bone plate is any shape useful in fixing bone fragments.

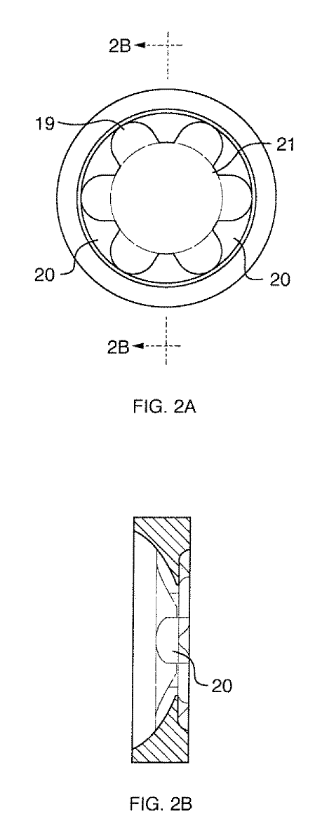

In an embodiment of the invention, and with reference to FIGS. 2a, 3a, and 4a, a fixation device 10 has at least one aperture 21 configured to accept a fixation element 1 to fasten fixation device 10 to a bone to be repaired. In an embodiment of the invention, fixation device 10 includes one to ten apertures 21. In another embodiment, fixation device 10 includes two to eight apertures 21. In another embodiment, fixation device 10 includes five apertures 21. In an embodiment of the present invention, aperture 21 is circular, square, oval, rectangular, triangular, or other regular shape. In another embodiment, aperture 21 is irregularly shaped. In another embodiment, where there is more than one aperture 21, each aperture 21 is the same or a different shape. In an embodiment of the invention, aperture 21 is defined by an aperture wall 23 that is formed by extension of aperture 21 through the entire thickness of fixation device 10, from the first outer surface 71 of fixation device 10 to the second outer surface 81 of fixation device 10.

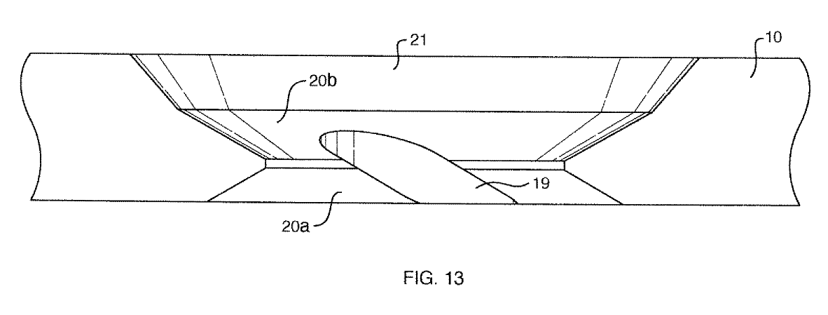

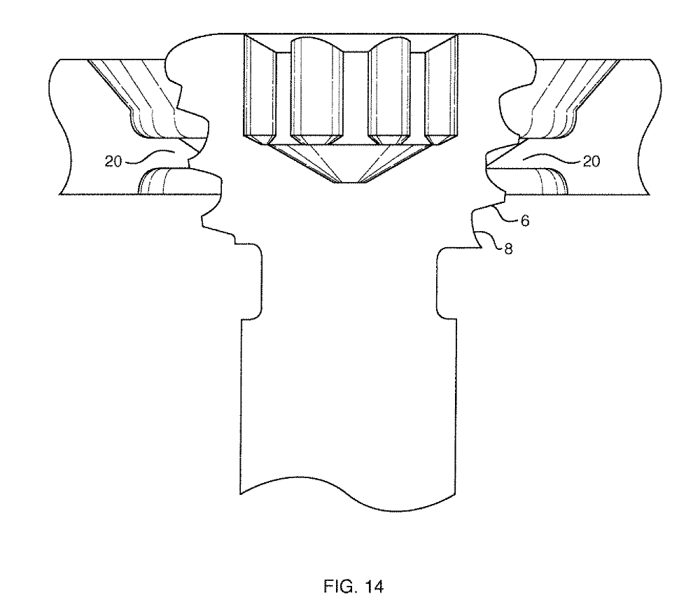

In an embodiment of the invention, fixation device 10 includes at least one tab slot 19 defined by adjacent tabs 20. In an embodiment of the invention, tab slot 19 has a substantially flat profile. In another embodiment, tab slot 19 has an angular profile. In an embodiment, and with reference to FIG. 13, fixation device 10 of the present invention includes an angular tab slot 19 to facilitate insertion of a fixation element 1.

In an embodiment of the present invention, fixation device 10 includes at least one tab 20. In an embodiment of the invention, there is a minimum of one tab. In another embodiment, there are 2 tabs; in another embodiment there are 3 tabs; in another embodiment there are 4 tabs; in another embodiment there are 5 tabs; in another embodiment there are 6 tabs; in another embodiment there are 7 tabs; in another embodiment there are 8 tabs; in another embodiment there are 9 tabs; in another embodiment there are 10 tabs; in another embodiment there are 11 tabs; in another embodiment there are 12 tabs; in another embodiment there are 13 tabs; in another embodiment there are 14 tabs. In an embodiment of the invention, tabs 20 form a “flower” pattern as shown, for example, in FIGS. 2a, 7, and 8.

With reference to FIGS. 2a, 4a, 7, and 8, in an embodiment of the invention, tabs 20 are of sufficient size and distance apart such that there is discontinuous circumferential engagement of the fixation element head threads 5 and tabs 20. In an embodiment of the invention, tab 20 is of sufficient width (i.e., thickness) to deflect enough to engage threads 5 and resist the pullout force of fixation element 1. In an embodiment of the invention, for larger fixation devices, the thickness and size of tab 20 will be larger than for smaller fixation devices because larger fixation elements 1 will require larger, stronger tabs in order to resist pullout force and in order to prevent fixation element 1 from driving through aperture 21 without locking into place with tabs 20. If tab 20 is too thick, fixation element 1 will not advance through aperture 21. In another embodiment, the geometry of tabs 20 (that is, spacing and shape of the tabs relative to one another) is optimized so that the insertion torque is similar for each kind of material (for example, stainless steel alloys or titanium).

In an embodiment of the invention, the ratio of the thickness of tab 20 to the pitch of threads 5 is from about 1:1 to about 1:4. In another embodiment, the ration is from about 1:1.5 to about 1:3. In another embodiment, the ratio is from about 1:2 to about 1:2.5. In another embodiment, the ratio is about 1:2 (see FIG. 14). In another embodiment, the ratio of the spacing between tabs 20 to the pitch of threads 5 is about 1:0.5 to about 1.5:1. In another embodiment, the ratio is about 1:1. These ratios will vary depending on the material; for example, tabs manufactured from steel will be thinner and narrower compared with tabs manufactured from titanium for a given size fixation element head thread.

In an embodiment of the invention, fixation device 10 and fixation element 1 are manufactured from the same material. In another embodiment, they are manufactured from different material. In an embodiment of the invention, tabs 20 are manufactured of the same or different material than fixation device 10. In an embodiment of the invention, tabs 20 are manufactured from stainless steel or other alloys or titanium or titanium alloys, or any other biocompatible material.

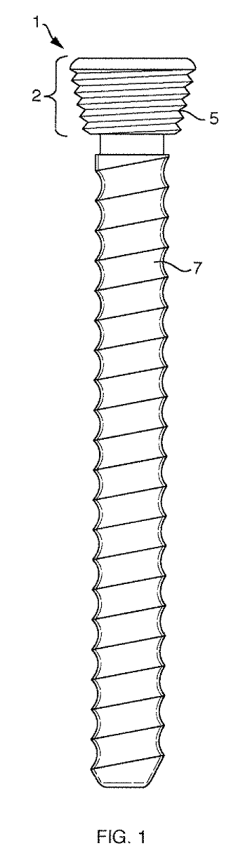

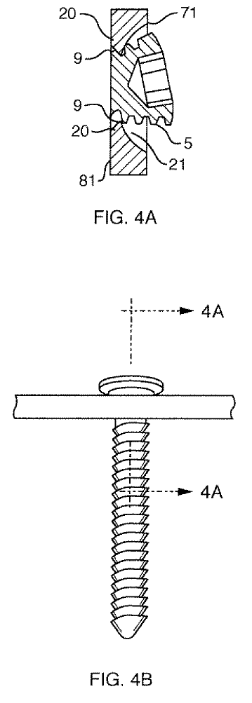

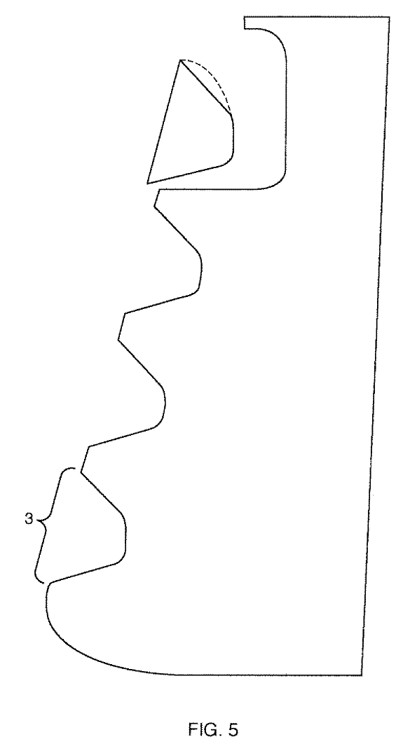

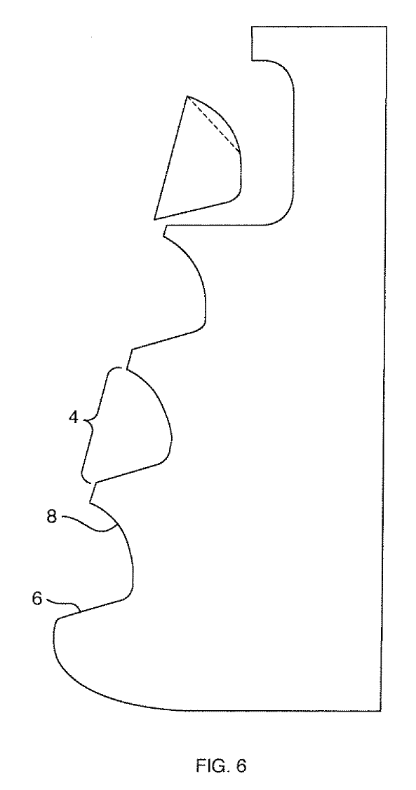

In an embodiment of the present invention, and with reference to FIGS. 1 and 4a, aperture 21 includes at least one tab 20 that engages threads 5 of a fixation element head 2 (e.g., a screw head). In an embodiment of the invention, aperture 21 includes at least two tabs 20, and tabs 20 are configured to deflect when engaged by threads 5 generally at the pitch angle or radius of threads 5 of fixation element head 2 when fixation element 1 is perpendicular to the plate. In one embodiment, this deflection upon engagement effectively produces a threaded interface between threads 5 and fixation device 10. In another embodiment, when fixation element 1 is removed from aperture 21, tabs 20 do not retract into their original position. In an embodiment of the invention, when the fixation element 1 is removed from aperture 21, tabs 20 retract into their original position.

In an embodiment of the invention, and with reference to FIG. 4a, tabs 20 result in points of contact 9 between fixation device 10 and threads 5 thus minimizing the potential for gauling between the fixation device and fixation element. This is in contrast to U.S. Pat. No. 6,974,461, claim 1 line 41, which describes a ridge, lip or edge. In an embodiment of the present invention, tabs 20 have a width sufficient to minimize the probability of gauling (cold welding) between threads 5 and the fixation device 10, for example, a bone plate. Gauling is the negative effect of the threads of a fixation element and the fixation device become welded together.

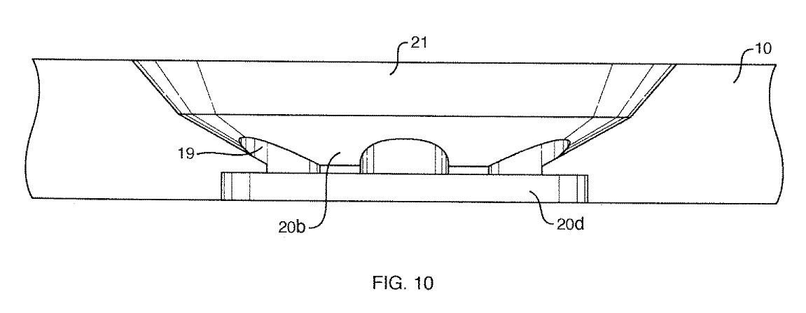

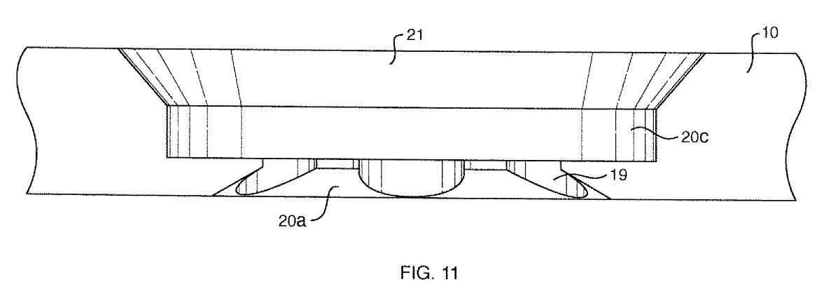

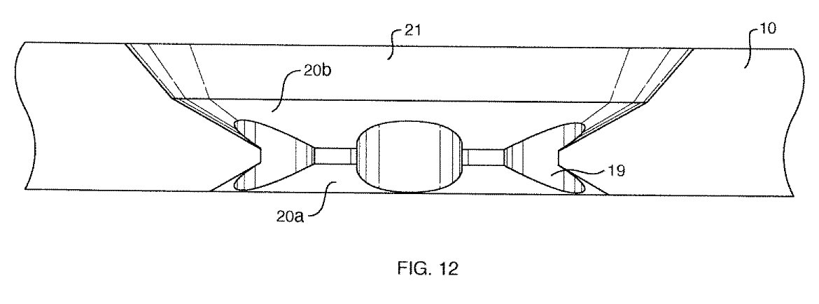

In an embodiment of the present invention, and with reference to FIGS. 10-12, tab 20 includes an angular surface 20(a) and/or 20(b) (i.e., a countersink). In another embodiment, tab 20 includes a countersink 20(a) or 20(b) and a substantially flat surface 20(c) or 20(d) (i.e., a counterbore). In an embodiment of the present invention, angular surface 20(a) or 20(b) of tab 20 aids the engagement of threads 5 on the fixation element head 2 to the tab 20. In an embodiment, angular surface 20(a) or 20(b) of tab 20 reduces the required insertion torque. In an embodiment, and with reference to FIG. 10, tab 20 includes a superior angular surface 20(b) and a substantially flat inferior surface 20(d). In an embodiment, and with reference to FIG. 11, tab 20 includes a substantially flat superior surface 20(c) and an inferior angular surface 20(a). In an embodiment, and with reference to FIG. 12, tab 20 includes a superior angular surface 20(a) and an inferior angular surface 20(b).

B. Fixation Element

In an embodiment of the invention, at least one fixation element, i.e., a fastener, is used with the fixation device. Referring to FIG. 1, in one embodiment, the fixation element 1 is selected from the group consisting of a screw, a nail, a peg, a bolt, or another fastener. In an embodiment of the invention, fixation element 1 is a bone screw. In another embodiment, fixation element 1 includes threads 5 on the head 2 of fastener 1, and threads 7 on the body of fixation element 1. In an embodiment of the invention, fixation element head 2 is unthreaded. In another embodiment of the present invention, fixation element head 2 is threaded. In an embodiment, the threaded profile of fixation element head 2 minimizes the insertion torque of the fixation element 1 into the fixation device 10. In another embodiment, any fixation element 1 is useful in accordance with the present invention, including those now known in the art and those that are future developed.

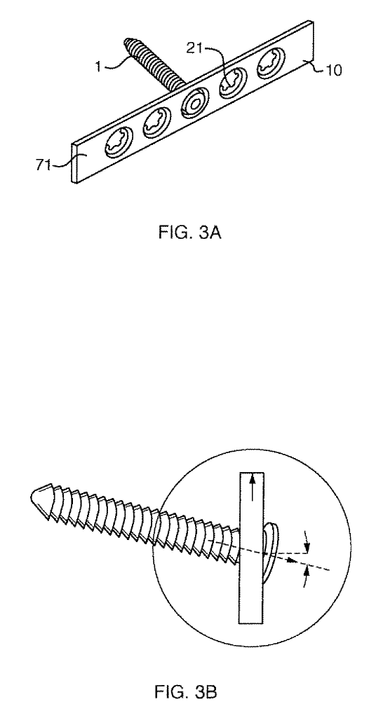

FIGS. 3a, 3b, and 4a illustrate the interface between tabs 20 and threads 5. In an embodiment of the invention, fixation element 1 is inserted into aperture 21 at an angle of up to 20 degrees relative to the fixation device. In another embodiment, fixation element 1 is inserted into aperture 21 at an angle of about 15 degrees relative to the fixation device.

The present invention also relates to a fixation element 1, having a particular thread design to maximize the functionality, design, and usefulness of such a fixation element with a fixation device 10 according to the present invention. In an embodiment of the invention, threads 5 are designed to deflect in any direction tabs 20 of fixation device 10 according to the present invention. In another embodiment of the invention, and with reference to FIG. 4a, the fixation element head thread profile minimizes the angle between threads 5 and contact points 9.

In an embodiment of the present invention, and with reference to FIG. 5, threads 5 have an angular profile 3 of from about 30 to about 75 degrees, and are generally a “V” shape. In another embodiment, threads 5 have an angular profile 3 of from about 40 to about 70 degrees. In another embodiment, threads 5 have an angular profile 3 of from about 50 to about 60 degrees. In another embodiment, threads 5 have an angular profile 3 of about 60 degrees.

In an embodiment of the present invention, and with reference to FIG. 6, the threads 5 have a radiused profile 4, and are in a general “U” shape. In an embodiment, threads 5 have a radius of from about 0.1 mm to about 2.0 mm. In another embodiment, the radius is from about 0.1 mm to about 1.5 mm. In another embodiment, the radius is from about 0.1 mm to about 1.0 mm. In another embodiment, the radius is from about 0.1 mm to about 0.5 mm. In another embodiment, the radius is from about 0.1 mm to about 0.4 mm. In another embodiment, the radius is about 0.3 mm. In an embodiment, radiused profile 4 includes a superior angular surface 6 and an inferior radiused profile 8. In an embodiment, there is less interference between threads 5 with a radiused profile 4 and tabs 20 of the fixation device 10. In one embodiment, threads 5 with a radiused profile 4 require less deflection of tabs 20. In another embodiment, threads 5 having a radiused profile 4 decreases the insertion torque required for advancing fixation element 1 into fixation device 10 as described above.

One advantage of the present invention over the prior art is that no threads are left in the fixation device 10 upon removal of a fixation element 1. In the prior art device and method, threads remain in the plate upon removal of a fixation element (see e.g., U.S. Pat. No. 6,974,461, which is herein incorporated by reference). Use of deflecting tabs 20 to engage threads 5 is also an advantage over the prior art, which uses deforming plate material to engage fixation element threads 5.

C. Fixation Lag Tool

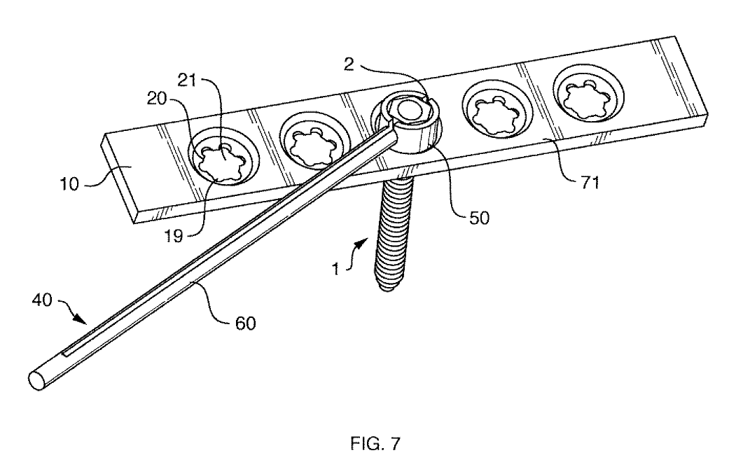

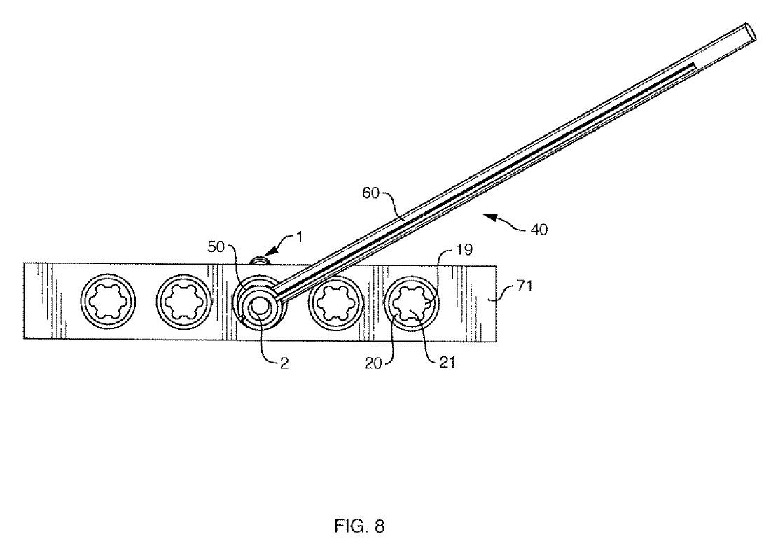

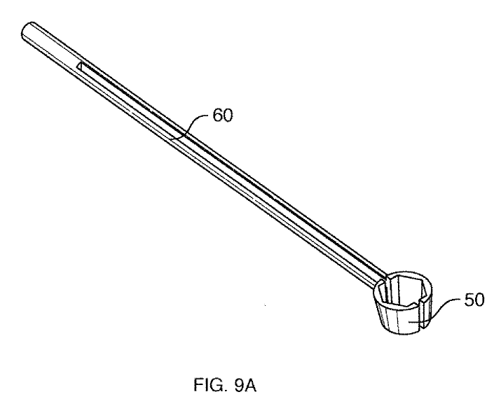

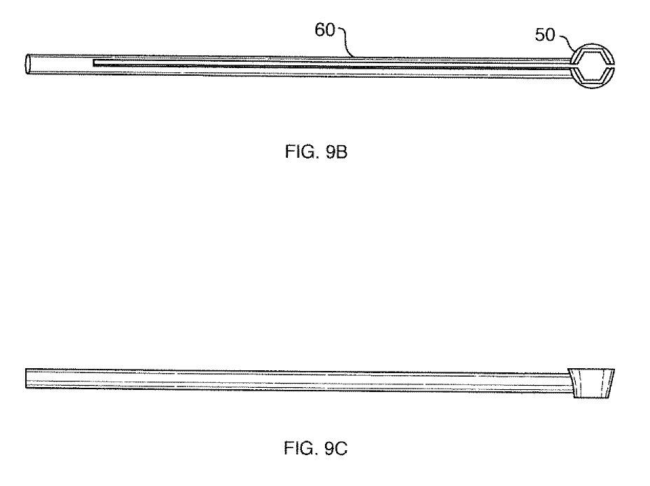

The present invention also relates to a tool that allows for induction of compression, or lag, across a fracture plane, and a method for inducing compression. In an embodiment of the invention, as shown in FIGS. 7-9, a lag tool 40 has a head 50 and a stem portion 60. In an embodiment of the invention, head portion 50 of the tool 40 is configured to fit between a fixation element head 2 and a fixation device 10, as shown in FIGS. 7 and 8. In another embodiment, fixation lag tool 40 is configured to allow fixation element 1 to continue to advance without head 2 coming into contact with the fixation device 10 until a desired compression is achieved across a fracture site being repaired.

In an embodiment of the invention, the method for inducing compression includes partially advancing a fully threaded or partially threaded locking fixation element 1 into an aperture 21 of fixation device 10; placing the lag tool 40 between fixation element head 2 and the fixation device 10; turning the fixation element 1 until the desired amount of compression is achieved across a fracture site being repaired; removing tool 40 and locking fixation element 1 into fixation device 10 as described above.

The present invention also relates to a locking fixation kit. The kit comprises a fixation lag tool 40 for inducing compression across a fracture plane, fixation device 10, and fixation element 1 according to the present invention.

It will be apparent to those skilled in the art that various modifications and variations can be made in the device of the present invention without departing from the scope of the invention. Thus, it is intended that the present invention cover the modifications and variations of the present invention. Any and all publications, patents, and patent applications are herein incorporated by reference in their entirety.

What is claimed is:

1. A fixation device comprising
a. a first outer surface;
b. a second outer surface;
c. an aperture defined at least in part by an aperture wall, the aperture extending from the first outer surface to the second outer surface; and
d. at least two tabs protruding from the aperture wall into the aperture,
wherein each of the at least two tabs are configured to form a locking securement with a fixation element disposed within the aperture.

2. The fixation device of claim 1, wherein at least one of the tabs includes a superior angular surface.

3. The fixation device of claim 2, wherein at least one of the tabs includes a substantially flat inferior surface.

4. The fixation device of claim 1, wherein at least one of the tabs includes an inferior angular surface.

5. The fixation device of claim 4, wherein at least one of the tabs includes a substantially flat superior surface.

6. The fixation device of claim 1, wherein at least one of the tabs includes a superior angular surface and an inferior angular surface.

7. The fixation device of claim 1, further comprising a tab slot to facilitate insertion of a fixation element.

8. The fixation device of claim 7, wherein the tab slot is an angular tab slot.

9. The fixation device of claim 7, wherein at least one of the tabs includes a superior angular surface.

10. The fixation device of claim 9, wherein at least one of the tabs includes a substantially flat inferior surface.

11. The fixation device of claim 7, wherein at least one of the tabs includes an inferior angular surface.

12. The fixation device of claim 11, wherein at least one of the tabs includes a substantially flat superior surface.

13. The fixation device of claim 7, wherein at least one of the tabs includes a superior angular surface and an inferior angular surface.

14. A fixation element comprising a threaded head, wherein the threaded head includes a radiused thread profile.

15. The fixation element of claim 14, wherein the threaded head includes a superior angular surface and an inferior radiused surface.

16. The fixation element of claim 14, wherein the radiused surface is in a range of from about 0.1 millimeter to about 2.0 millimeters.

17. A locking bone fixation system comprising:
a. the fixation device of claim 1; and
b. at least one fixation element configured to engage the tabs and to lock into the fixation device.

18. The locking bone fixation system of claim 17, wherein the fixation element comprises a threaded head having a superior surface and an inferior surface, wherein the superior surface includes an angular thread profile and the inferior surface includes a radiused thread profile.

19. The locking bone fixation system of claim 17, wherein the fixation element is configured to reduce interference upon engaging the tabs.

20. A locking bone fixation system comprising:
a. the fixation device of claim 7; and
b. at least one fixation element configured to engage the tabs and to lock into the fixation device.

21. The locking bone fixation system of claim 20, wherein the fixation element comprises a threaded head having a superior surface and an inferior surface, wherein the superior surface includes an angular thread profile and the inferior surface includes a radiused thread profile.

22. The locking bone fixation system of claim 20, wherein the fixation element is configured to reduce interference upon engaging the tabs.

23. A method for inducing compression across a fracture plane, the method comprising:
a. partially advancing a fixation element having a head into an aperture of a locking fixation device;
b. placing a lag tool between the fixation element head and the fixation device;
c. turning the fixation element head until a desired compression is achieved across the fracture plane; and
d. removing the lag tool and locking the fixation element into the fixation device.
